(19) United States
(12) Patent Application Publication
(10) Pub. No.: US 2023/0372173 A1
(43) Pub. Date: Nov. 23, 2023

(54) AIR TREATMENT

(71) Applicants: Scott Elrod, Lake Jackson, TX (US); Chad McClintick, Leawood, KS (US)

(72) Inventors: Scott Elrod, Lake Jackson, TX (US); Chad McClintick, Leawood, KS (US)

(21) Appl. No.: 18/040,607

(22) PCT Filed: Aug. 4, 2020
(86) PCT No.: PCT/US20/44840

§ 371 Date: Feb. 3, 2023

(51) Int. Cl. A61G 10/02; A61G 10/00; A61L 9/20

(52) U.S. Cl. CPC A61G 10/02; A61G 10/005; A61L 9/20; A61L 2209/12

(57) ABSTRACT

Devices, systems, and methods to treat an environment. The device includes a scent control material source. The device can also include a dehumidifier, humidifier, and one or more fans. The scent control material device is configured to output a scent control material (e.g., an oxidant) into the environment to reduce pathogens, dispose of scent molecules and their sources, and otherwise treat the environment. The device can include a controller communicatively coupled to the scent control material source, dehumidifier, humidifier, and one or more fans. The controller can implement operational programs dictating at least one output of the device. The device can also function in a cyclical or periodic mode in which an output of the device can be varied to more effectively treat the environment. In examples, the controller can operate in conjunction with a remote control to regulate output parameters of the device.

### BACKGROUND

Infected persons can transmit contaminants by expiring air with contaminants into their environment. Viruses and bacteria can be breathed, spat, or coughed as a mode of transmission. Medical professionals wear masks and other personal protective equipment to prevent professional-to-patent and patient-to-professional transmission of contaminants. However, some procedures may expose those involved to higher risks that others.

Transmission of contaminants can cause illness in medical professionals, patients, and those they come in contact with. Such illnesses may cause interruption in medical care and spread disease to additional people. Accordingly, contact with air expired from others may be undesirable to medical professionals, patients, and other occupants of an environment.

### SUMMARY

Embodiments of the invention relate to devices, systems, and methods for treating an environment to eliminate or reduce undesirable compounds.

A system for treating an environment according to at least some embodiments is disclosed. The system includes an enclosure configured to fit over at least a head of a patient. The enclosure of the system includes a fluid impermeable barrier sized and shaped to fit over at least the head of the patient and including an outer surface and an inner surface defining an interior region facing the patient. The system includes a vacuum source fluidly connected to the interior region via the fluid impermeable barrier. The system includes a treatment source fluidly connected to one or more of the vacuum source or the fluid impermeable barrier, the treatment source being configured to treat air from the interior region.

A system for treating an environment according to at least some embodiments is disclosed. The system includes an enclosure configured to fit over at least a head of a patient. The enclosure of the system includes a framework sized and shaped to fit over at least the head of the patient and a fluid impermeable barrier disposed on the framework, the fluid impermeable barrier including an outer surface and an inner surface defining an interior region facing the patient. The system includes a vacuum source fluidly connected to the interior region via the fluid impermeable barrier. The system includes a treatment source fluidly connected to one or more of the vacuum source or the fluid impermeable barrier, the treatment source being configured to treat air from the interior region.

A method of treating expired air is disclosed. The method includes covering at least the face of a patient with an enclosure configured to retain air expired from the patient in an interior region thereof. The method includes placing the interior region under negative pressure. The method includes receiving expired air from the patent within the interior region. The method includes treating the expired air to neutralize one or more contaminants therein.

A system for treating an environment according to at least some embodiments is disclosed. The system includes an enclosure configured to fit over at least a head of a patient and at least a portion of an operation table or chair. The enclosure of the system includes a framework sized and shaped to fit over at least the head of the patient and a fluid impermeable barrier disposed on the framework, the fluid impermeable barrier including an outer surface and an inner surface defining an interior region facing the patient. The enclosure of the system includes one or more access ports disposed on the fluid impermeable barrier, the one or more access ports being configured to resealably allow access to the interior region from outside of the fluid impermeable barrier. The system includes a vacuum source fluidly connected to the interior region via the fluid impermeable barrier. The system includes a treatment source fluidly connected to one or more of the vacuum source or the fluid impermeable barrier, the treatment source being configured to treat air from the interior region.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a block diagram of a system for treating an environment, according to at least some embodiments.

FIG. 2A is an isometric view of an enclosure in a first configuration, according to at least some embodiments.

FIG. 2B is an isometric view of the enclosure of FIG. 2A in a second configuration, according to at least some embodiments.

FIG. 3 is a schematic of a system for treating air in an environment, according at least some embodiments.

FIG. 4 is a schematic of a system for treating air in an environment, according at least some embodiments.

FIG. 5 is an isometric view of an enclosure, according to at least some embodiments.

FIG. 6 is a front view of the enclosure of FIG. 5, according to at least some embodiments.

FIG. 7 is a side view of the enclosure of FIG. 5, according to at least some embodiments.

FIG. 8 is a top view of the enclosure of FIG. 5, according to at least some embodiments.

FIG. 9 is an isometric view an enclosure in use in a system for treating an environment, according to at least some embodiments.

FIG. 10 is a block diagram of a controller for executing any of the example methods disclosed herein, according to an embodiment.

FIG. 11 is a flow diagram of a method of treating expired air, according to at least some embodiments.

### DETAILED DESCRIPTION

Embodiments of the invention relate to devices, systems, and methods for treating air expired from a patient within an environment. The air expired from a patient can be trapped in an interior region of an enclosure disposed over the face of the patient. The air in the environment can be treated to neutralize contaminants therein. For example, an enclosure can be disposed on the face of a patient undergoing a medical procedure. As the patient breaths or is intubated, air expired by the patient into the enclosure can be trapped, treated, and removed, thereby preventing any of the contaminants therein from reaching the medical professionals treating the patient. The contaminants such as bacteria, fungi, viruses, or the like in the expired air can be treated with ultraviolet light, oxidants, or heat to neutralize the contaminants.

In embodiments, a system can be utilized to prevent the exposure of expired air and to treat the expired air to neutralize contaminants therein. As such, the term “treat,” “treated,” “treating,” or “treatment” can refer to applying material or controlling conditions to neutralize contaminants in the expired air. “Neutralize” can refer to killing, deactivating, or otherwise rendering the contaminants unable to infect, spread, or multiply. For example, treating a contaminant may include rendering a virus or bacteria unable to spread by applying ultraviolet light, heat, or an oxidant (e.g., ozone) to the contaminant. Accordingly, the systems, methods, and devices herein can be used to prevent the spread of viruses, bacteria, or the like from a patient to medical professionals or vice versa. The systems, devices, and methods herein also neutralize the air expired from a patient, including all of the contaminants therein, allowing the air to be later expelled into the environment without spreading the contaminants (e.g., infecting others).

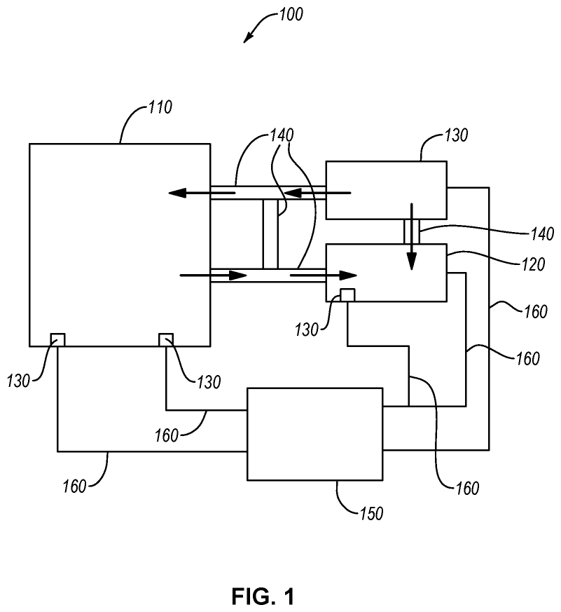

FIG. 1 is a block diagram of a system 100 for treating an environment, according to an embodiment. The system 100 includes an enclosure 110, a vacuum source 120, and at least one treatment source 130. The enclosure 110, the vacuum source 120, and the at least one treatment source 130 may be fluidly connected to each other via one or more conduits 140. The system 100 may include a controller 150 to control one or more of the vacuum source 120 or the treatment source(s) 130. The controller 150 may be operably coupled to the vacuum source 120 and the treatment sources 130 via one or more connections 160. In practice, the enclosure 110 may be placed over at least the face of a patient to capture any air expired from the patient. The vacuum source 120 creates negative pressure to in the enclosure and removes the expired air therefrom. The treatment source(s) 130 are located in positions within the system 100 to treat the expired air in one or more of the enclosure 110, the vacuum source 120, or the conduits 140. Accordingly, the system 100 captures expired air and treats the expired air to neutralize any contaminants therein.

The enclosure 110 is sized and shaped to fit over at least the head of a patient. The enclosure 110 may include a framework and a fluid impermeable barrier disposed on the framework. The framework is sized and shaped to fit over at least the head of the patient. The framework may be a hard frame (e.g., metal, plastic, or wooden frame) or a soft frame (e.g., inflatable or foam frame). The fluid impermeable barrier is disposed on and supported by the framework so that the fluid impermeable barrier does not impede breathing of the patient. The fluid impermeable barrier includes an inner surface and an outer surface defining an interior region in the enclosure which faces the patient. For example, at least the patient's face may be disposed in the interior region. The fluid impermeable barrier is formed form an at least partially transparent polymer as disclosed in more detail below. As the patient expires air, the expired air is contained within the interior region, thereby preventing the air from reaching the medical professionals (e.g., doctors, nurses, dentists, technicians, paramedics) and them from breathing the expired air. The interior region of the enclosure 110 is fluidly coupled to the vacuum source 120 via the fluid impermeable barrier.

The vacuum source 120 may include a vacuum with a container configured to hold and filter the air received therein. The vacuum source 120 may apply negative pressure in the interior region of the enclosure 110, such as to remove air therefrom. The air may be pulled into the vacuum source 120 via conduit 140 (e.g., collection conduit). The vacuum source 120 may include a vacuum, a vacuum line (e.g., in a hospital room), or the like. The vacuum or negative pressure supplied by the vacuum source 120 prevents the expired air from going outside of the interior region.

The at least one treatment source 130 treats the expired air to neutralize one or more contaminants therein. The at least one treatment source 130 may be fluidly connected to one or more of vacuum source 120, the conduits 140, or the fluid impermeable barrier (e.g., in the interior region). The at least one treatment source 130 may be disposed in the interior region of the enclosure 110, outside of the enclosure 110 in a separate apparatus (e.g., ozone generator), in the vacuum source 120, in the conduit(s) 140, or combinations thereof. The at least one treatment source 130 may include one or more of an ultraviolet light source, an oxidant source (e.g., ozone source), a heat source, or the like. For example, an ozone generator may be fluidly connected to the interior region of the enclosure 110 via a section of conduit 140 (e.g., input conduit). Accordingly, the ozone generator may input ozone into the enclosure to treat the expired air of the patient within the interior region. To prevent overexposure of the patient to ozone, the input of the ozone (e.g., conduit 140) may be located at or near the intake (e.g., conduit 140) of the vacuum source 120. Accordingly, as the expired air is removed from the interior region it is exposed to ozone to neutralize contaminants therein.

Suitable oxidant generators may include ozone generators, ion generators, or the like. Suitable ozone generators may include plasma arc ozone generators, such as the Orion or HR230 ozone generators from Ozonics (located in Mason City, Iowa, USA) or the like. The oxidant generator may include a fan to propel the oxidant from the oxidant generator. The oxidant generator may direct a selected amount of oxidant output per unit time, such as according to a program stored in the generator or the controller 150. For example, a base oxidant emission rate may be at least 50 mg of oxidant (e.g., ozone) per hour (“mg/hr”), such as about 50 mg/hr to about 1000 mg/hr, about 100 mg/hr to about 500 mg/hr, about 500 mg/hr to about 1000 mg/hr, less than about 500 mg/hr, less than about 300 mg/hr, more than 500 mg/hr, about 500 mg/hr to about 15,000 mg/hr, about 1,000 mg/hr to about 10,000 mg/hr, less than 15,000 mg/hr, less than 10,000 mg/hr, or less than 5,000 mg/hr. In examples, the oxidant generator and/or the controller may direct emission of oxidant for a duration of at least 5 seconds (s), such as 5 seconds to 1 hour, 30 seconds to 20 minutes, 1 minute to 40 minutes, less than an hour, or less than 20 minutes. In some examples, the oxidant emission rates, amounts, and durations herein can be sufficient to provide exposure of the contaminants (e.g., virus, bacteria, etc.) to the oxidant of at least about 0.1 min-mg/m³, such as about 0.1 mg-min/m³ to about 20 mg-min/m³, about 0.2 mg-min/m³ to about 15 mg-min/m³, about 0.3 mg-min/m³ to about 10 mg-min/m³, about 0.1 mg-min/m³ to about 5 mg-min/m³, about 3 mg-min/m³ to about 7 mg-min/m³, about 7 mg-min/m³ to about 12 mg-min/m³, less than about 20 mg-min/m³, or less than 12 mg-min/m³.

The at least one treatment source 130 may include one or more ultraviolet lights sources. Ultraviolet light may be used to neutralize contaminants in the expired air, such as ultraviolet light at a wavelength expected to neutralize one or more contaminants. The wavelength of UV light may be at least 100 nm, such as between 100 nm and 320 nm, 150 nm and 300 nm, 200 nm and 280 nm, 200 nm and 225 nm, 225 nm and 250 nm, 250 nm and 280 nm, 280 nm to 320 nm, 320 nm to 400 nm, less than 400 nm, less than 300 nm, or more than 200 nm. As shown, ultraviolet light sources (labeled as treatment sources 130) may be disposed on the enclosure 110 such as within the interior region. The duration of the exposure to UV light may be the same as disclosed above. The conduit 140 fluidly connected to the vacuum source 120 may include one or more UV light sources therein. The canister or vacuum storage tank of the vacuum source may include the one or more UV light sources therein. Accordingly, the expired air may be treated as it is removed from the interior region or stored in the vacuum source 120.

In some examples, a heat source (e.g., infrared heater, microwave generator, or the like) may be disposed in the conduit 140 or the vacuum source 120. The heat source may heat the contaminants to a temperature at or above a lethal temperature for viruses, bacteria, or mold. In some example, the heat source may be contained in a heat proof portion of the conduit 140 (away from the patient). The heat source may heat the expired air to a temperature of at least 60° C. to neutralize contaminants therein, such as at 60° C. to 100° C., 100° C. to 150° C., 150° C. to 200° C., less than 200° C., or less than 100° C.

The one or more conduits 140 may fluidly connect the enclosure 110 to the vacuum source 120, the enclosure 110 to a treatment source 130, or a first conduit to a second conduit, such as to treat air in a conduit 140 fluidly connected to the vacuum source 120. One or more of the conduits 140, or portions thereof, may be formed of polymer(s), metal(s), glass, or the like. For example, the conduit 140 may include one or more of polyamides, polycarbonates, polyetherimides, polyesters, polyethylene (e.g., LDPE, HDPE), polypropylenes, polytetrafluoroethylene, polystyrenes, polysulfones, polyurethanes, polyether ether ketone, plastomers, elastomers (e.g., thermoplastic elastomers), silicone, polyvinyl chloride (e.g., chlorinated PVC), or the like. The conduits 140 may include one or more portions that are constructed to withstand the treatment applied by the treatment source 130. For example, the conduit 140 may include a metal section where the heat source is located. The conduit 140 may be constructed of a material that is resistant to oxidants such as ozone or UV light (e.g., polycarbonates or HDPE). One or more portions of the conduits 140 may be flexible or rigid.

The conduits 140 fluidly connect an external vacuum source 120 to the interior region of the enclosure 110. As shown, the conduits 140 may fluidly connect an external treatment source 130 to the interior region of the enclosure 110. The conduits 140 may fluidly connect the external treatment source 130 to the vacuum source 120. The conduits 140 may fluidly connect a conduit 140 from the external treatment source 130 to the interior region with another conduit 140 from the interior region to the vacuum source 120, such as to input treatment material (e.g., ozone) therein.

While not shown, system 100 may include one or more conduits for venting the treated air, such as into the internal environment (e.g., within the room or building) or external environment (e.g., outside of the building). For example, the vacuum source 120 may include a vacuum pump operably coupled to multiple air treatment devices where the vacuum pump exhausts the treated air out a flue or stack of the building.

One or more of the vacuum source 120 or the treatment sources 130 may be controlled by the controller 150. The controller 150 may be operably coupled to the vacuum source 120 and the at least one treatment source 130 via one or more connections 160. The one or more connections 160 may be wired connections or wireless connections. The controller 150 may be equipped (e.g., programmed) to independently operate the vacuum source 120 and the at least one treatment source 130. For example, the controller 150 may include activation buttons and/or operational programs for each of the at least one treatment sources 130 and the vacuum source 120. Additionally, the controller 150 may include adjustment mechanisms for one or more of the at least one treatment source 130 or the vacuum source 120. In such examples, the controller 150 may be used to initiate, increase, decrease, or terminate application of treatment (e.g., production of heat, light, or oxidants) or vacuum. The controller 150 may be located on one or more of the enclosure 110, the vacuum source 120, or the at least one treatment source 130. The controller 150 may be remote from one or more of the enclosure 110, the vacuum source 120, or the at least one treatment source 130, such as on a remote control, a tablet (e.g., as an application), a computer or the like. The controller 150 is described in more detail below.

One or more components of the system 100 may be omitted in some embodiments. For example, the controller 150 may be omitted. In such examples, the vacuum source 120 and the treatment source(s) 130 may be controlled via controls (e.g., power switch) thereon, respectively.

While FIG. 1 depicts a general embodiment of a system for treating air in an environment, further refinements and embodiments for the components of the system 100 may be used in systems for treating air in an environment.

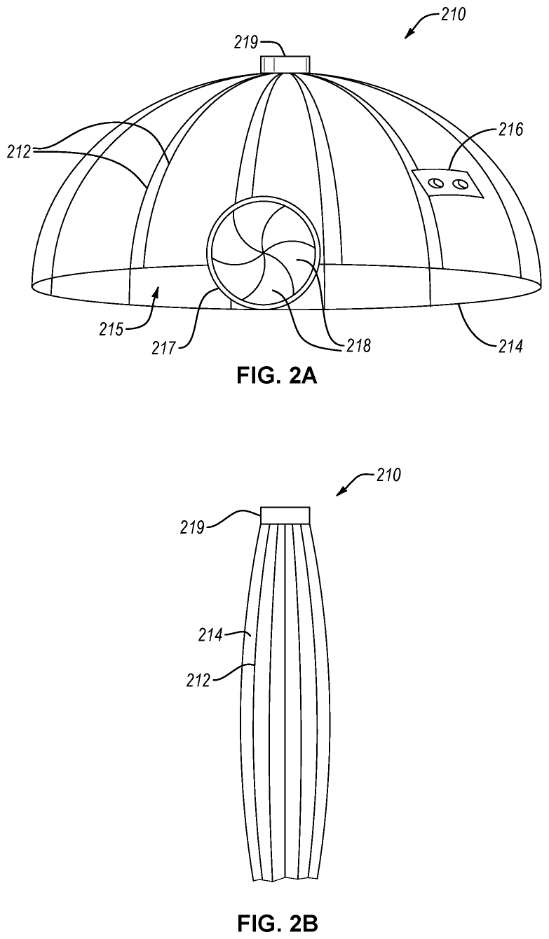

FIG. 2A is an isometric view of an enclosure 210 in a first configuration, according to at least some embodiments. The enclosure 210 may be sized to fit over at least the nose and mouth of a patient, at least the head of the patient, or at least the head and upper body of the patient. The enclosure 210 may be similar or identical to the enclosure 110 in one or more aspects. For example, the enclosure 210 includes framework 212 and fluid impermeable barrier 214. The framework 212 may be similar or identical to any of the frameworks disclosed herein, in one or more aspects. The framework 212 may include a plurality of members to form a rigid or semi-rigid support. For example, the framework 212 may include rigid or semi-rigid members, such as metal, plastic, inflatable, foam, etc., members. The individual members of the framework 212 may be connected provide at least a semi-rigid structure for the fluid impermeable barrier 214 to sit upon. The framework 212 may form a frame or cage. As shown, the framework 212 includes a plurality of members (e.g., ribs) connected at, and extending from, a central point or hub 219. The framework 212 may be foldable, collapsible, bendable, or otherwise manipulable. The framework 212 may include one or more of runners, supports, struts, ribs, or the like. The hub 219 may be shaped as or include a handle.

The fluid impermeable barrier 214 is disposed on the framework 212. The fluid impermeable barrier 214 may be similar or identical to any of the fluid impermeable barriers disclosed herein, in one or more aspects. The fluid impermeable barrier 214 includes a fluid impermeable material such as a polymer material. For example, the fluid impermeable barrier 214 may include one or more of polyamides, polycarbonates, polyetherimides, polyesters, polyethylene (e.g., LDPE, HDPE), polypropylenes, polytetrafluoroethylene, polystyrenes, polysulfones, polyurethanes, polyether ether ketone, acrylics, plastomers, elastomers (e.g., thermoplastic elastomers), silicone, polyvinyl chloride (e.g., chlorinated PVC), or the like, or copolymers of any of the same. One or more portions of the fluid impermeable barrier 214 may be at least partially transparent to allow medical professionals to see inside of the interior region 215 therein. For example, the transparent polymer may include a transparent form of such as being formed from a transparent polymer. The fluid impermeable barrier 214 may be formed of a material that is resistant to UV light damage and/or oxidation (e.g., acrylonitrile, methyl methacrylate. The fluid impermeable barrier 214 may include a UV light resistant coating (e.g., TiO₂) or oxidant resistant coating thereon to prevent damage to the underlying material of the fluid impermeable barrier 214. The fluid impermeable barrier 214 may be flexible.

The fluid impermeable barrier 214 is disposed on the framework 212. For example, the fluid impermeable barrier 214 may have one or more portions (e.g., pockets, tabs, etc.) which accommodate the framework 212 therein or thereon. Accordingly, manipulation of the framework 212 may also manipulate the fluid impermeable barrier 214. Such manipulation may quickly open or close the enclosure 210. As shown, the enclosure 210 may be generally dome shaped when deployed in the first configuration. The enclosure 210 may be collapsible, akin to an umbrella. For example, the members of the framework 212 may be connected to the hub 219 and fold thereabout.

FIG. 2B is an isometric view of the enclosure 210 of FIG. 2A in a second configuration, according to at least some embodiments. As shown in FIG. 2B, the framework 212 and the fluid impermeable barrier 214 thereon may be folded about the hub 219 after use to store the enclosure 210.

Returning to FIG. 2A, the enclosure may include a means for connecting one or more of a vacuum source or treatment source, such as via one or more conduits. For example, the enclosure 210 may include a manifold 216 thereon. The manifold 216 includes one or more connections for fluidly connecting the interior region of the enclosure 210 to one or more conduits. Such connections may be attachments for cylindrical conduits. In some examples, the manifold 216 may be omitted and one or more orifices for conduits may be present in the fluid impermeable barrier 214.

The enclosure 210 may include one or more access ports 217. The one or more access ports 217 may be located in the fluid impermeable barrier 214. The one or more access ports 217 may provide resealable access to the interior region 215 from outside of the fluid impermeable barrier 214. The one or more access ports 217 may be a door, a window, a glove access, a diaphragm or the like. As shown, access port 217 is configured as a diaphragm, such as a diaphragm with resilient blades 218 or leafs that bend responsive to mechanical force. For example, the blades 218 of the diaphragm may be disposed in a planar first orientation and contact each other to substantially seal the external environment from the interior region 215 and when a medical professional pushes the blades 218 they bend to a second orientation to allow the medical professional's hand(s) and arm(s) to enter the interior region. Upon removing their hand(s) and arm(s), the blades 218 return to the first orientation, thereby substantially resealing the interior region 215. In some examples, the diaphragm may include an iris diaphragm. In some examples, the at least one access port 217 may include a hinged door. In some examples, the at least one access port 217 may include a glove attached (e.g., sealed) to the fluid impermeable barrier 214 through which a medical professional's hands (and arms) may be inserted into the interior region, in the glove. In some examples, the at least one access port 217 may include a puncturable polymer or rubber membrane disposed over the at least one access port 217. The at least one access port 217 may include overlapping rubber or polymer film sheets, which may be manipulated to open and close.

Any number of access ports may be located on the enclosure 210, such as at least one access port 217 on each side of the enclosure 210, or two, three, or four access ports on at least one respective side or surface of the enclosure 210. Accordingly, medical professionals may be able to perform procedures such as intubation, extubation, surgery, or the like while the enclosure is disposed over the head of the patient.

While shown as separate members, in some examples, the framework 212 may be included in the fluid impermeable barrier 214. Put another way, the fluid impermeable barrier 214 may include one or more portions that are semi-rigid or rigid. Accordingly, one or more portions of the fluid impermeable barrier 214 may act as at least a portion of the framework.

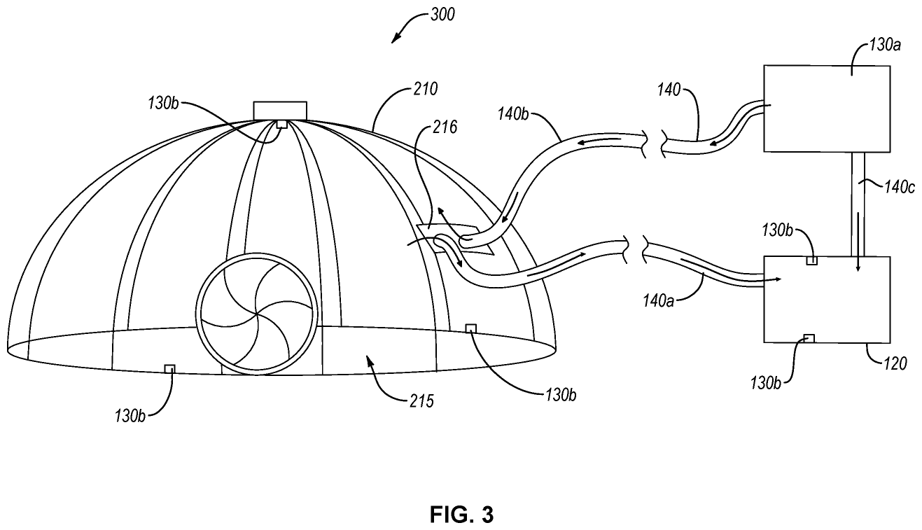

FIG. 3 is a schematic of a system 300 for treating air in an environment, according to at least some embodiments. The system 300 includes the enclosure 210, vacuum source 120, treatment sources 130a and 130b, and conduits 140. As shown, the interior region of the enclosure 210 is operably coupled to the vacuum source 120 and the treatment source 130a (e.g., ozone generator) via conduits 140a and 140b. For example, the conduit 140a may extend from the manifold 216 to the treatment source 130a and conduit 140a may extend from the manifold 216 to the vacuum source 120. Another conduit 140c may fluidly connect the treatment source 130a with the vacuum source 120. Accordingly, the treatment source 130a may be utilized to input oxidants, such as ozone, into one or more of the interior region 215 or the vacuum source (e.g., a canister, storage tank, or filter therein).

In some examples, one or more of the treatment sources 130b may be disposed in the system 300 such as in the enclosure 210 and/or in the vacuum source 120 (e.g., in a canister or filter therein). For example, treatment sources 130b may include UV light sources. The system 300 allows users to treat air expired from a patient into the interior region using one or more of UV light or oxidants (e.g., ozone) and in the vacuum source using UV lights or oxidants.

By locating the input for the treatment source 130a near the intake for the vacuum source 120, oxidant may be input into the interior region 215 to treat air as it is removed from the interior region. Such a configuration limits or may even eliminate a patient's exposure to the oxidants. While some examples are configured to input oxidants into the interior region of the enclosure, other embodiments may not expose the patient to oxidants at all, such as when oxidants are input into the intake conduit or the vacuum source 120.

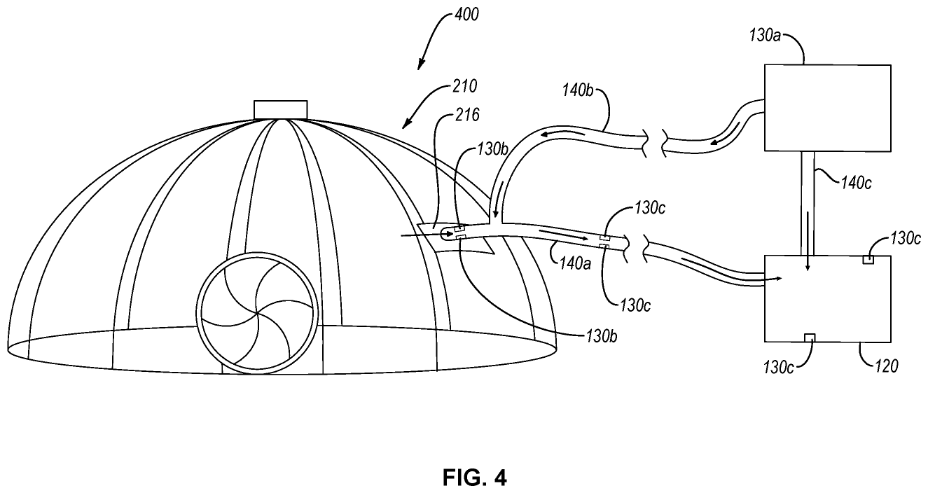

FIG. 4 is a schematic of a system 400 for treating air in an environment, according at least some embodiments. The system 400 includes the enclosure 210, vacuum source 120, treatment sources 130a-c, and conduits 140. As shown, the interior region of the enclosure 210 is operably coupled to the vacuum source 120 via conduits 140a. For example, the conduit 140a extends from the manifold 216 the manifold 216 to the vacuum source 120. Conduit 140b may extend form the treatment source 130a (e.g., ozone or other oxidant generator) to the conduit 140a. Another conduit 140c may fluidly connect the treatment source 130a with the vacuum source 120. Accordingly, the treatment source 130a may be utilized to input oxidants, such as ozone, into one or more of the conduit 140a or the vacuum source 120 (e.g., a canister, storage tank, or filter therein). In such examples, only the expired air inside of the conduit 140 or vacuum source 120 is exposed to the oxidants.

In some examples, one or more of the treatment sources 130b and/or 130c may be disposed in the system 400 such as in the vacuum source 120 (e.g., in a canister or filter therein). For example, treatment sources 130b may include UV light sources may be disposed in the conduit 140a. Treatment sources 130c may be disposed in the conduit 140a or in the vacuum source 120. The treatment sources 130c may include heat sources as disclosed herein. The heat source 130c may be located remotely from the enclosure 210 to prevent exposure to the patient. Accordingly, the system 400 allows users to treat air expired from a patient in the interior region using one or more of UV light, heat, or oxidants (e.g., ozone) and in the conduit 140a or vacuum source 120.

While shown as a dome shaped in FIGS. 2A-4, enclosures may be configured in any suitable shape to cover at least the nose and mouth or face of a patient. For example, enclosures may be sized and shaped to fit around at least a portion of an operation table or chair, such as a surgical table, a dental chair, or the like.

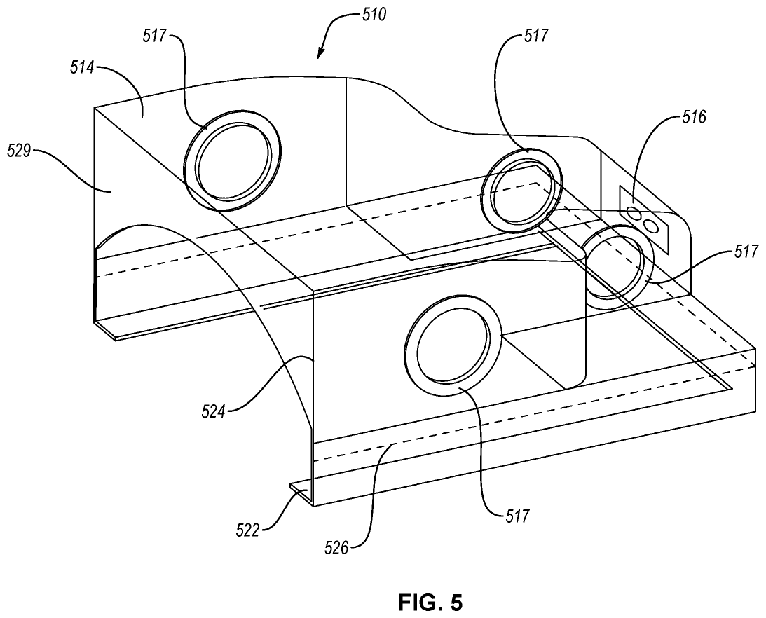

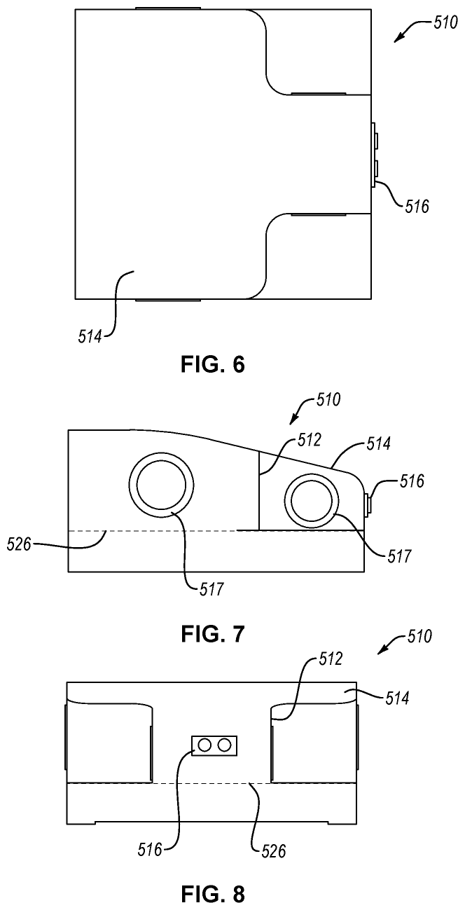

FIG. 5 is an isometric view of an enclosure 510, according to at least some embodiments. FIG. 6 is a front view of the enclosure 510 of FIG. 5, according to at least some embodiments. FIG. 7 is a side view of the enclosure 510 of FIG. 5, according to at least some embodiments. FIG. 8 is a top view of the enclosure 510 of FIG. 5, according to at least some embodiments. The enclosure 510 includes the framework 512 and the fluid impermeable barrier 514. The framework 512 may be similar or identical to the framework 212, in one or more aspects. The fluid impermeable barrier 514 may be similar or identical to the fluid impermeable barrier 214, in one or more aspects. The enclosure 510 is sized and shaped to fit around at least a portion of an operation table or chair. As shown, the enclosure 510 is sized and shaped to fit over a surgical table and to accommodate at least a portion of the upper body (e.g., face or head) of the patient therein. The enclosure 510 includes a lower portion 522, an upper portion 524, and a reusable connection 526. The lower portion 522, upper portion 524, and a reusable connection 526 may be used to seal the patient in an interior region of the enclosure 510.

The lower portion 522 may include portions of one or more of the framework 512 or the fluid impermeable barrier 514. The lower portion 522 may be sized and shaped to accommodate (e.g., fit around) and hold on to one or more portions of the operation table or chair. For example, the lower portion 522 may be sized and shaped to fit around the mattress and supporting frame of a surgical table, with a retention means thereon. The retention means may be an elastic cuff, draw-string, or the like around the bottom of the lower portion 522 to synch or otherwise attach the lower portion 522 under and around the operation table or chair to hold the lower portion 522 thereon during use. For example, the lower portion 522 may fit around the operation table or chair and be held thereon with an elastic band around the lower extent thereof (e.g., akin to a fitted sheet 10).

The lower portion 522 may include at least some of the framework 512 therein. For example, the framework 512 may be present in the lower portion 522 in the vertically extending corners thereof. Alternatively, the lower portion may not include framework 512, such as when the fluid impermeable barrier 514 includes one or more rigid or semi-rigid portions thereof.

In some examples, the lower portion 522 may have attachments for attaching to the operation table or chair, such as snaps, hook and loop fasteners, buttons, magnets, or the like. In such examples, the attachments may attach to complementary attachments on the side or back side of the operation table or chair so that the lower portion 522 remains fixed thereto. At least a portion of the upper extent of the lower portion 522 may include the reusable connection(s) 526 thereon. The reusable connection 526 is used to connect the lower portion 522 to the upper portion 524. For example, the reusable connection 526 may extend along at least a portion of the uppermost region of the lower portion 522 and a lowermost region of the upper portion 524, such as along the sides of the enclosure 510 or around the entire enclosure 510. The reusable connection 526 may substantially seal the enclosure 510, such as seal the lower portion 522 to the upper portion 524. The reusable connection 526 may include a zipper, snaps, hook and loop fasteners, or the like.

At least a portion of the lower region of the upper portion 524 may include a portion of the reusable connection 526 thereon. For example, the lowermost extent of the upper portion 524 may include the portion of the zipper, snaps, button holes, hook and loop fasteners, or the like corresponding to a portion of the reusable connection 526 on the lower portion 522.

The upper portion 524 may include portions of one or more of the framework 512 or the fluid impermeable barrier 514. The upper portion 524 includes the portion of the fluid impermeable barrier 514 that extends over the patient's body, such as over at least the face, head or upper body of the patient. The upper portion 524 may be contoured to fit the shape of the human body, such as tapering at the shoulder region to the head region. As shown, such a configuration brings the fluid impermeable barrier 514 closer to the patient in areas where the patient is expected to be located during use. As shown in FIGS. 5-7, the front of the upper portion 524 may taper from a greater thickness at the chest region to a narrower thickness at the head region. As shown in FIG. 8, the chest and abdominal region in the central area of the upper portion 524 may have greater thickness than the arm regions at the sides of the upper portion 524.

The upper portion 524 may include at least some of the framework 512 therein. For example, the framework 512 may be present in the upper portion 524 in the vertically extending corners thereof. In some examples, the framework 512 may be omitted in favor of one or more rigid or semi-rigid portions of the fluid impermeable barrier 514. For example, the upper portion 524 may not include framework 512, such as when the fluid impermeable barrier 514 includes one or more rigid or semi-rigid portions thereof.

The sides of the upper portion 524 may include one or more access ports 517 therein. The access ports 517 may be similar or identical to the access ports 217 disclosed herein, in one or more aspects. In some examples, one or more access ports 517 may be located on the front or top of the upper portion 524. The access ports 517 may be strategically located to provide a medical professional access to the patient adjacent to the access ports 517, such as near the head, chest, abdomen, shoulders, etc.

The bottom end 529 of the upper portion 524 (shown on the left side of FIG. 5) may conform to the anatomy of the patient. For example, the bottom end 529 of the upper portion 524 may include a section of fluid impermeable barrier 514 sized and shaped to fit against the body of the patient, such as to at least partially seal the interior region of the enclosure 510. The bottom end 529 may contact the patient to form an at least partial seal against the patient. The air in the interior region may be prevented from passing out of the via the bottom end 529. The fluid impermeable barrier in the bottom end 529 may include elastic or another material to produce tension or a seal against a patient.

In some examples (not shown), the front of the upper portion 524 may taper to meet the chest, neck, abdomen or the like of the patient, to form an at least partial seal against the patient. In some examples, the enclosure may enclose the entire patient, such as over the entire operation table or chair.

The upper portion 524 may include the manifold 516. The manifold 516 may be similar or identical to the manifold 216, in one or more aspects. The manifold 516 may be located in the lower portion 522 in some examples.

One or more portions of the enclosure 510 may be collapsible, such as with folding framework, inflatable framework, or the like.

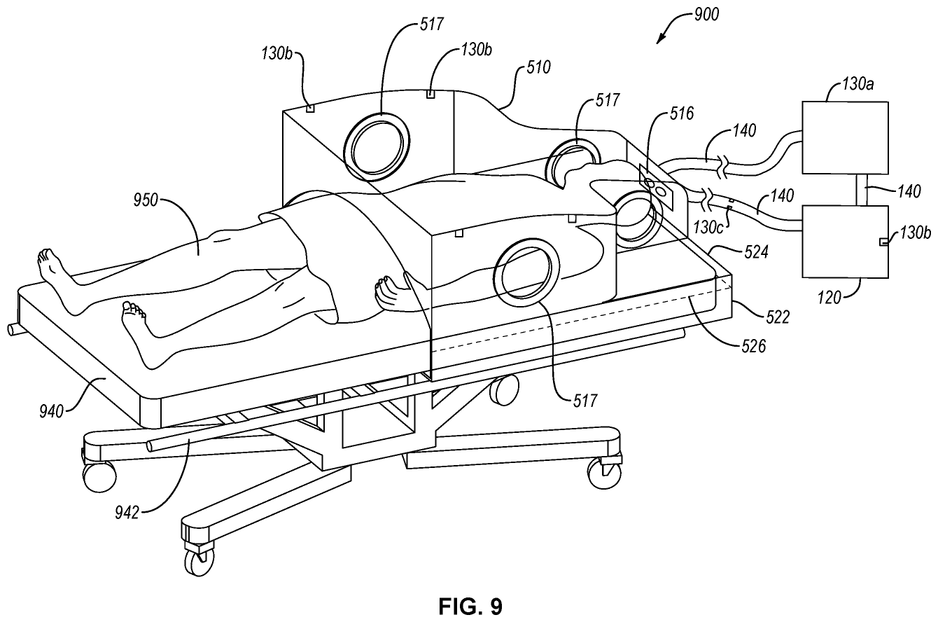

FIG. 9 is an isometric view an enclosure 510 in use in a system 900 for treating an environment, according to at least some embodiments. The system includes the enclosure 510, the one or more treatment sources 130a-c, and the vacuum source 120. The enclosure 510 may be disposed on an operation table 940 over a patient 950. The lower portion 522 may be disposed around the sides and/or under the bed 942 of the operation table 940. The upper portion 524 may be disposed around the sides and over the bed 942 and the patient 950. The lower portion 522 and the upper portion 524 may be connected via the reusable connection 526 to seal the patient 950 within the interior region of the enclosure 510. After connecting the lower portion 522 to the upper portion 524, medical professionals may perform one or more procedures, such as intubation, via the access ports 517. After the procedure(s), the upper portion 524 may be removed from the lower portion 522 via the reusable connection (e.g., unzipping the zipper), more additional procedures may be carried out, and/or the patient 950 may be removed from the operation table 940.

The vacuum source 120 may be operably coupled to the interior region of the enclosure 510 via the manifold 516 and conduit 140 (e.g., inlet conduit). The vacuum source 120 may be used to place the interior region under negative pressure to remove any air expired from the patient. In some examples, the enclosure 510 may include one or more one-way valves, in the access ports 517 or fluid impermeable barrier to prevent the enclosure from collapsing under negative pressure.

As shown, one or more treatment sources 130a-c may be used in conjunction with the enclosure 510. For example, treatment source 130a (e.g., oxidant source) may be operably coupled to one or more of the enclosure 510, the vacuum source 120, or the inlet conduit to treat the air therein with oxidants such as ozone. Treatment sources 130b (e.g., UV light source) may be disposed in the interior region of the enclosure, in a canister of the vacuum source 120, or in the conduits 140. Treatment sources 130c (e.g., heat source) may be disposed in the canister of the vacuum source 120 or in the conduits 140. The treatment sources 130a-c are used to treat the air expired from the patient to neutralize any contaminants therein, as disclosed herein.

The system 900 may include a controller 150 (FIG. 1) operably coupled to the treatment sources 130a-c and the vacuum source 120. The treatment sources 130a-c and the vacuum source 120 may be controlled via the controller 150. The controller 150 may control application of vacuum in the interior region and application of treatment from the treatment source(s).

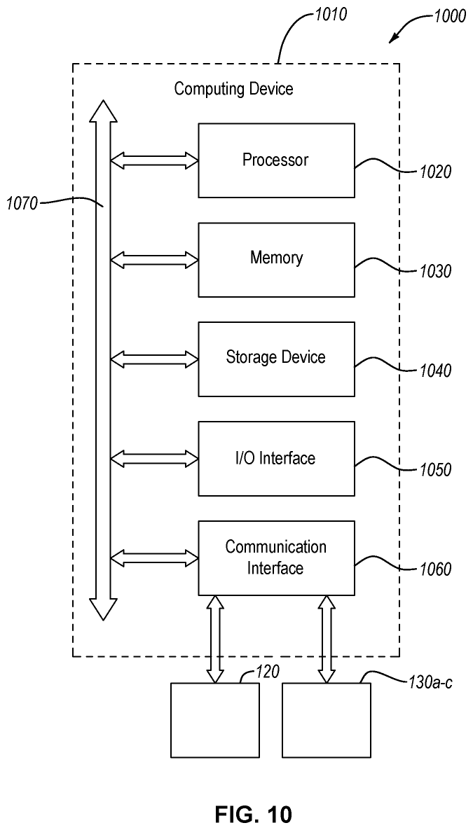

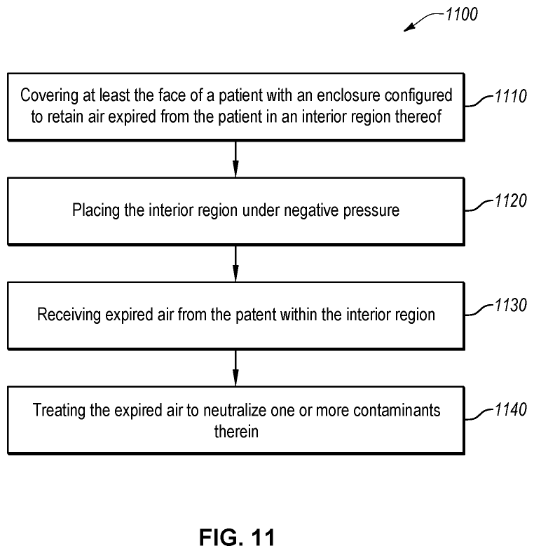

Any of the example controllers or systems disclosed herein may be used to carry out any of the example methods disclosed herein. FIG. 10 is a block diagram of a controller 1000 for executing any of the example methods disclosed herein, according to an embodiment. The controller 1000 may implement any of the example methods disclosed herein, such as method 1100 (FIG. 11). The controller 1000 includes at least one computing device 1010. The at least one computing device 1010 is an exemplary computing device that may perform one or more of the acts described herein, such as in the method 1100. The at least one computing device 1010 may include one or more servers, one or more computers (e.g., desk-top computer, lap-top computer), one or more mobile computing devices (e.g., smartphone, tablet, etc.), or one or more custom computing systems assembled to execute proprietary functions (e.g., remote control). The computing device 1010 may comprise at least one processor 1020, memory 1030, a storage device 1040, an input/output (“I/O”) device/interface 1050, and a communication interface 1060. In examples, the computing device 1010 may be sized to fit in another device, such as on the manifold or fluid impermeable barrier of an enclosure.

While an example computing device 1010 is shown in FIG. 10, the components illustrated in FIG. 10 are not intended to be limiting of the controller 1000 or computing device 1010. Additional or alternative components may be used in some examples. Further, in some examples, the controller 1000 or the computing device 1010 may include fewer components than those shown in FIG. 10. In some examples, the at least one computing device 1010 may include connections to a plurality of computing devices, such as a server farm, computational network, or cluster of computing devices. Components of computing device 1010 shown in FIG. 10 are described in additional detail below.

In some examples, the processor(s) 1020 includes hardware for executing operational programs or instructions (e.g., instructions for carrying out one or more portions of any of the methods disclosed herein), such as those making up a computer program. For example, to execute operational programs or instructions, the processor(s) 1020 may retrieve (or fetch) the operational instructions from an internal register, an internal cache, the memory 1030, or a storage device 1040 and decode and execute them. In particular examples, processor(s) 1020 may include one or more internal caches for data such as oxidant output parameters or voltage amounts correlated to UV light or heat output parameters. As an example, the processor(s) 1020 may include one or more instruction caches, one or more data caches, and one or more translation lookaside buffers (TLBs). Operational instructions in the instruction caches may be copies of instructions in memory 1030 or storage device 1040. In some examples, the processor 1020 may be configured (e.g., include programming stored thereon or executed thereby) to carry out one or more portions of any of the example methods disclosed herein.

In some examples, the processor 1020 performs any of the acts disclosed herein such as in method 1100 cause one or more portions of the computing device 1010 or controller 1000 to perform at least one of the acts disclosed herein. Such a configuration may include one or more operational programs (e.g., computer program products) or application software that are executable by the at least one processor 1020. For example, the processor 1020 may automatically select an operational program responsive to receiving a command (e.g., button input from a user).

The at least one computing device 1010 (e.g., a server, remote control, or remote input device) may include at least one memory storage medium (e.g., memory 1030 and/or storage device). The computing device 1010 may include memory 1030, which is operably coupled to the processor(s) 1020. The memory 1030 may be used for storing data, metadata, application software, and operational programs for execution by the processor(s) 1020. The memory 1030 may include one or more of volatile and non-volatile memories, such as Random Access Memory (RAM), Read Only Memory (ROM), a solid state disk (SSD), Flash, Phase Change Memory (PCM), or other types of data storage. The memory 1030 may be internal or distributed memory. The computing device 1010 may include the storage device 1040 having storage for storing data or instructions (e.g., application software, oxidant or light output parameters, and operational programs). The storage device 1040 may be operably coupled to the at least one processor 1020. In some examples, the storage device 1040 may comprise a non-transitory memory storage medium, such as any of those described above. The storage device 1040 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 1040 may include removable or non-removable (or fixed) media. Storage device 1040 may be internal or external to the computing device 1010. In some examples, storage device 1040 may include non-volatile, solid-state memory. In some examples, storage device 1040 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 1030 and/or storage device 1040 (e.g., memory storage medium(s)) may store one or more databases thereon. At least some of the databases may be used to store one or more of inputs, command instructions, or output parameters of the vacuum source 120 or treatment source(s) 130a-c.

In some examples, one or more of application software, operational programs (e.g., programs controlling output from the vacuum source or treatment sources), or any other data, may be stored in a memory storage medium such as one or more of the at least one processor 1020 (e.g., internal cache of the processor), memory 1030, or the storage device 1040. In some examples, the at least one processor 1020 may access (e.g., via bus 1070) the memory storage medium(s) such as one or more of the memory 1030 or the storage device 1040. For example, the at least one processor 1020 may receive and store the data (e.g., look-up tables) as a plurality of data points in the memory storage medium(s). The at least one processor 1020 may execute programming stored therein adapted access the data in the memory storage medium(s) to perform any of the acts disclosed herein.

The computing device 1010 also includes one or more I/O devices/interfaces 1050, which are provided to allow a user to provide input to, receive output from, and otherwise transfer data to and from the computing device 1010. These I/O devices/interfaces 1050 may include a mouse, keypad or a keyboard, a touch screen, camera, optical scanner, network interface, web-based access, modem, a port, other known I/O devices, or a combination of such I/O devices/interfaces 1050. The touch screen may be activated with a stylus or a finger.

The I/O devices/interfaces 1050 may include one or more devices for presenting output to a user, including, but not limited to, a graphics engine, a display (e.g., a display screen or monitor), one or more output drivers (e.g., display drivers), a user interface, one or more audio speakers, and one or more audio drivers. In certain examples, I/O devices/interfaces 1050 provide graphical data to a display for presentation to a user. The graphical data may be representative of one or more graphical user interfaces and/or any other graphical content as may serve a particular implementation.

The computing device 1010 may further include a communication interface 1060. The communication interface 1060 may include hardware, software, or both. The communication interface 1060 may provide one or more interfaces for communication (such as, for example, packet-based communication) between the computing device 1010, a remote control, a remote input device, the vacuum source 120, the treatment source(s) 130a-c, and one or more additional (e.g., remote) computing devices or one or more networks. For example, communication interface 1060 may include a network interface controller (NIC) or network adapter for communicating with an Ethernet or other wire-based network or a wireless NIC (WNIC) or wireless adapter for communicating with a wireless network, such as a WI-FI.

Any suitable network and any suitable communication interface 1060 may be used. For example, computing device 1010 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 1000 or computing device 1010 may communicate with a wireless PAN (WPAN) (such as, for example, a BLUETOOTH WPAN), a WI-FI network, a WI-MAX network, a cellular telephone network (such as, for example, a GSM network), or other suitable wireless network or a combination thereof. Computing device 1010 may include any suitable communication interface 1060 for any of these networks, where appropriate.

The computing device 1010 may include the bus 1070. The bus 1070 may include hardware, software, or both that couples components of computing device 1010 to each other. For example, bus 1070 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

It should be appreciated that any of the examples of acts described herein, such as in the method 1100 below, may be performed by and/or at the controller 1000 or computing device 1010 thereof. As noted the computing device 1010 may be sized, shaped, and otherwise fit one or within the devices or systems (e.g., remote control, etc.) disclosed herein. The operational programs may be stored and/or executed by the one or more of the controller 1000 or the computing device 1010 therein.

FIG. 11 is a flow diagram of a method 1100 of treating expired air, according to at least some embodiments. The method 1100 includes the act 1110 of, covering at least the face of a patient with an enclosure configured to retain air expired from the patient in an interior region thereof. The method 1100 includes the act 1120 of, placing the interior region under negative pressure. The method 1100 includes the act 1130 of, receiving expired air from the patient within the interior region. The method 1100 includes the act 1140 of, treating the expired air to neutralize one or more contaminants therein. In examples, any of the acts 1110-1140 may be omitted, combined with other acts, or performed in a different order than presented. For example, the act of placing the interior region under negative pressure may be omitted. Any of the components of the systems for treating air within an environment disclosed herein may be used to perform the method 1100.

The method 1100 includes the act 1110 of covering at least the face of a patient with an enclosure configured to retain air expired from the patient in an interior region thereof. Covering at least the face of a patient with an enclosure configured to retain air expired from the patient in an interior region thereof may include using any of the enclosures disclosed herein. For example, the enclosure may include a framework sized and shaped to fit over at least the head of the patient and a fluid impermeable barrier disposed on the framework, the fluid impermeable barrier including an outer surface and an inner surface defining the interior region facing the patient. In some examples, the framework may be omitted, such as when the fluid impermeable barrier includes a semi-rigid or rigid material.

Covering at least the face of a patient with an enclosure may include placing the enclosure over the nose and mouth of the patient. Covering at least the face of a patient with an enclosure may include placing the enclosure over the upper body or head of the patient. Covering at least the face of a patient with an enclosure may include placing the enclosure around a portion of an operation table or chair having the upper body of the patient thereon and sealing the enclosure around at least a portion of the upper body of the patient using the reusable connection.

The method 1100 includes the act 1120 of, placing the interior region under negative pressure. Placing the interior region under negative pressure may include applying a vacuum in the interior region via a vacuum source operably coupled to the enclosure. Put another way, placing the interior region under negative pressure includes using a vacuum source operably coupled to the enclosure to suction the expired air from the enclosure. The vacuum source may include any of the vacuum sources disclosed herein. For example, the vacuum source may include one or more of a vacuum hose or a vacuum canister. Placing the interior region under negative pressure may include activating the vacuum source via an activation switch, button, or other means or a controller operably coupled thereto.

The method 1100 includes the act 1130 of, receiving expired air from the patient within the interior region. Receiving expired air from the patent within the interior region may include preventing air expired from the patient from leaving the interior region. Receiving expired air from the patent within the interior region may include intubating the patient. Receiving expired air from the patent within the interior region may include extubating the patient.

The method 1100 includes the act 1140 of, treating the expired air to neutralize one or more contaminants therein. Treating the expired air to neutralize one or more contaminants therein may include using any of the treatment devices disclosed herein. Treating the expired air to neutralize one or more contaminants therein may include treating the expired air in the interior region, in the conduits, or in the vacuum source.

Treating the expired air to neutralize one or more contaminants therein may include inputting ozone into one or more of the interior region, the vacuum source, or the conduit(s). Treating the expired air to neutralize one or more contaminants therein may include exposing the expired air to ozone with a concentration of at least 0.5 parts per million for at least 30 minutes, such as 0.5 ppm to 20 ppm (e.g., 5-20 ppm) for 30 minutes to 2 hours. Treating the expired air to neutralize one or more contaminants therein may include irradiating one or more of the interior region, the interior of the conduits, or the vacuum source with ultraviolet radiation at a wavelength (e.g., 100 nm to 320 nm) selected to neutralize one or more of bacteria, viruses, or fungi. Treating the expired air in the vacuum source may include one or more of exposing the expired air to ozone or ultraviolet radiation at a wavelength between 100 nm and 320 nm in the one or more of a vacuum hose or vacuum canister of the vacuum source.

One or more of placing the interior region under negative pressure and treating the expired air to neutralize one or more contaminants therein may be carried out responsive to an operational program from a controller. For example, the operational program may initiate and control application of negative pressure and initiate and control application of one or more treatments from one or more treatment sources according to present operational parameters (e.g., amount of vacuum, amount of oxidant produces, amount of UV light produced, amount of heat, etc.). The operational programs may include machine readable and executable instructions for controlling any aspect of the vacuum and treatments, such as timing and intensity. Such operational programs may be stored in the controller or in the vacuum source or treatment sources themselves.

The method 1100 may include performing a medical procedure via one or more access diaphragms disposed on the enclosure. For example, the method 1100 may include performing surgery or a dental procedure on the patient. The method 1100 may include removing the enclosure. For example, removing the enclosure may include removing the upper portion from the lower portion via the reusable connection (e.g., unzipping, unsnapping, or the like). Removing the enclosure may include taking the enclosure off of the mouth and nose or face of the patient.

In some examples, the method 1100 may include sealing the enclosure without a person therein and treating the interior region with one or more treatment devices to sanitize the interior region. For example, ozone may be pumped into the interior region to clean the interior region. Accordingly, the enclosure may be sanitized after use for possible reuse. In some examples, the method 1100 may include sealing the enclosure and treating the interior region prior to performing a procedure to ensure that the interior region is sanitized (e.g., contaminants therein are neutralized).

In some examples, the devices and systems disclosed herein may be utilized to capture (and treat) gases expired from the patient outside of the operation room, such as in a recovery room. For example, the method 1100 may be carried out on a patient after a procedure, such as during recovery, to capture gases expired from the patient after the procedure is completed. Patients who have been intubated, sedated, or given anesthetic(s), such as gases (e.g., nitrous oxide or the like), may continue to exhale unsolubilized gases for a time after the gases are administered and procedure is complete. Accordingly, it can also be important to protect medical professionals from contamination of anesthetic gases, viruses, bacteria, or the like while the patient is recovering, after a procedure. Notably, long term effects of breathing in even minimal concentrations of nitrous oxide or other anesthetic gases may be harmful to medical professionals. Thus, capturing gases expired from the patient may be carried out for at least 10 minutes after a procedure is completed, such as at least 20 minutes, at least 30 minutes, at least 45 minutes, or at least an hour.

Capturing gases expired from the patient may include carrying out one or more of the acts 1110-1140 (or any additional acts of the method 1000) after a medical or dental procedure is completed, such as in the operating room or a recovery room (e.g., a room outside of the operating room). In some examples, the method 1100 may include moving the patient to a recovery room and utilizing the enclosure to capture gases expired from the patient. Capturing gases expired from the patient may include repeating one or more of the acts 1110-1140 in the recovery room after carrying out the acts 1110-1140 in the operating room.

As nitrous oxide or other contaminants are collected from the patient, the contaminants may be neutralized. For example, nitrous oxide may be exposed to UV light or combined with ozone to break the nitrous oxide down to nitric oxide, nitrogen, and oxygen. Likewise fluorinated anesthetics such as enflurane, isoflurane, desflurane, sevoflurane, halothane, etc. may be neutralized prior to venting into the environment. In addition to protecting medical professionals, such neutralization may also prevent or reduce atmospheric ozone depletion.

The systems and methods herein prevent medical professionals from being exposed to contaminants in the air expired from patients and treats the air to neutralize any contaminants therein. The treatments are capable of being applied in the interior region or in the vacuum source or conduits to prevent patient exposure. Further, the systems and methods disclosed herein allow the expired air to be recirculated into the local environment (e.g., operating room or office), after treatment, without exposing people in the local environment to non-neutralized contaminants or limiting the exposure thereto.

In some examples, the endpoint values disclosed herein may be approximate values, which may vary by 10% or less from the precise endpoint value given. In such examples, the term “about” or “substantially” may indicate the approximate values.

Aspects of any of the examples disclosed herein may be used with aspects of any other examples, disclosed herein without limitation.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

1. A system for treating an environment, the system comprising:
an enclosure configured to fit over at least a head of a patient, the enclosure including:
a framework sized and shaped to fit over at least the head of the patient; and
a fluid impermeable barrier disposed on the framework, the fluid impermeable barrier including an outer surface and an inner surface defining an interior region facing the patient;
a vacuum source fluidly connected to the interior region via the fluid impermeable barrier; and
a treatment source fluidly connected to one or more of the vacuum source or the fluid impermeable barrier, the treatment source being configured to treat air from the interior region.

2. The system of claim 1 wherein the enclosure has a domed shape.

3. The system of claim 1 wherein the framework includes a metal frame.

4. The system of claim 1 wherein the framework includes one or more inflatable members.

5. The system of claim 1 wherein the framework is foldable.

6. The system of claim 1 wherein the fluid impermeable barrier includes a transparent polymer.

7. The system of claim 1 further comprising one or more access ports disposed in the fluid impermeable barrier.

8. The system of claim 1 wherein the enclosure is sized and shaped to fit around at least a portion of an operation table or chair.

9. The system of claim 1 wherein the enclosure includes an upper portion, a lower portion, and a reusable connection between the upper portion and the lower portion.

10. The system of claim 9 wherein the reusable connection includes a zipper.

11. The system of claim 1 wherein the treatment source includes one or more of an oxidant source or a UV-light source.

12. The system of claim 1 wherein the vacuum source is fluidly coupled to the interior region via a collection conduit and the treatment source is fluidly coupled to the collection conduit and configured to treat air passing therethrough.

13. The system of claim 1 wherein the treatment source is fluidly coupled to the interior region via an input conduit and the treatment source includes an oxidant generator configured to input oxidants into the interior region.

14. The system of claim 1 wherein the treatment source is fluidly coupled to the interior region via an input conduit and the treatment source includes an oxidant generator configured to input oxidants into the interior region.

15. The system of claim 1, further comprising a controller operably coupled to the vacuum source and treatment source, the controller being configured to control application of vacuum in the interior region and application of treatment from the treatment source.

16. A method of treating expired air, the method comprising:
covering at least the face of a patient with an enclosure configured to retain air expired from the patient in an interior region thereof;
placing the interior region under negative pressure;
receiving expired air from the patent within the interior region; and
treating the expired air to neutralize one or more contaminants therein.

17.-24. (canceled)

25. The method of claim 16 wherein treating the expired air to neutralize one or more contaminants therein includes inputting ozone into the interior region.

26. The method of claim 16 wherein treating the expired air to neutralize one or more contaminants therein includes irradiating the interior region with ultraviolet radiation at a wavelength selected to neutralize one or more of bacteria, viruses, or fungi.

27. The method of claim 26 wherein the wavelength is between 100 nm and 320 nm.

28.-32. (canceled)

33. A system for treating an environment, the system comprising:
an enclosure configured to fit over at least a head of a patient and at least a portion of an operation table or chair, the enclosure including:
a framework sized and shaped to fit over at least the head of the patient; and
a fluid impermeable barrier disposed on the framework, the fluid impermeable barrier including an outer surface and an inner surface defining an interior region facing the patient; and
one or more access ports disposed on the fluid impermeable barrier, the one or more access ports being configured to resealably allow access to the interior region from outside of the fluid impermeable barrier;
a vacuum source fluidly connected to the interior region via the fluid impermeable barrier; and
a treatment source fluidly connected to one or more of the vacuum source or the fluid impermeable barrier, the treatment source being configured to treat air from the interior region.

34.-45. (canceled)
